(19) United States
(12) Patent Application Publication
(10) Pub. No.: US 2007/0280939 A1
(43) Pub. Date: Dec. 6, 2007

(54) Prostatic Acid Phosphatase Antigens

(76) Inventors: Robert Charles Rees, Sheffield (GB); Stephanie Eugenie Brigitte McArdle, Swadincote (GB); Richard John Parkinson, Nottingham (GB)

Correspondence Address: BARNES & THORNBURG LLP, 11 SOUTH MERIDIAN, INDIANAPOLIS, IN, 46204, US

(21) Appl. No.: 10/592,928

(22) PCT Filed: Mar. 17, 2005
(86) PCT No.: PCT/GB05/01030

§ 371 Date: Jun. 21, 2007

(30) Foreign Application Priority Data
Mar. 17, 2004 (GB) 0406054.7

(51) Int. Cl. A61K 39/395; A61K 31/7105; A61K 31/711; A61P 35/00; C07K 16/40; C12N 15/12; C12N 15/63; C12N 9/16; G01N 33/566; G01N 33/68; G01N 33/574; C12Q 1/68; C12N 5/10; C12N 15/55; C07K 7/00; C07K 16/18; A61K 38/08; A61K 38/10

(52) U.S. Cl. 424/139.1; 424/141.1; 435/320.1; 435/325; 435/6; 435/7.92; 436/501; 436/86; 514/14; 514/15; 514/44; 530/326; 530/328; 530/387.9; 536/23.5

(57) ABSTRACT

The invention relates to peptides from Prostatic Acid Phosphatase (PAP), especially those identified as PAP.135 and PAP.161. The nucleic acid molecules encoding the peptides, antibodies against the peptides, and the use of such peptides, nucleic acids and antibodies in immunotherapy, vaccines and assays are also included in the scope of the invention.

The invention relates to peptides from Prostatic Acid Phosphatase (PAP), especially those identified as PAP.135 and PAP.161. The nucleic acid molecules encoding the peptides, antibodies against the peptides, and the use of such peptides, nucleic acids and antibodies in immunotherapy, vaccines and assays are also included in the scope of the invention.

Prostate cancer is one of the most common cancers world-wide, causing illness and early death in a high proportion of men affected with this disease. Treatment options for patients with advanced or metastatic disease are limited, and additional therapeutic modalities required. Immunotherapy aims to produce immune-mediated killing in a tissue specific manner, targeting antigens that are present exclusively or up-regulated on tumour cells but not on normal cell types. A number of potential antigen targets for prostate cancer immunotherapy have been identified, including prostate-specific antigen (PSA), prostate stem cell antigen (PSCA), prostate specific membrane antigen (PSMA), the homeobox gene NKx3.1, and the prostatic acid phosphatase (PAP). In order to develop vaccination strategies for cancer therapy, it is necessary to identify antigenic peptide epitopes that are expressed by tumour cells, and recognised by cytotoxic T-lymphocytes (CTL). CTL epitope based vaccine approaches offer potential benefits over whole antigen based vaccines; the response can be focussed towards epitopes that are known to be good targets for CTL-mediated cytotoxicity. Furthermore, epitopes derived from proteins implicated in inducing and maintaining neoplastic transformation can be selected, whereas the administration of the entire parent protein would be potentially hazardous. The identification of CTL epitopes is also important in formulating multi-epitope vaccines to allow the targeting of multiple tumour specific antigens simultaneously.

While the stimulation of CTL is the primary goal of anti-tumour cellular immunotherapy, it has become clear that T-helper lymphocytes play a critical role in the generation and the maintenance of specific CTL responses directed towards MHC-associated peptide antigens expressed by the tumour. Moreover, CD4+ T cells have been shown to be able to control tumour growth independently of CTL killing (Greenberg PD 1991) and to be important in maintaining CTL memory in the absence of CD4+ T cells; dendritic cells fail to become fully activated and are therefore not able to stimulate a CTL response. The inclusion of T-helper epitopes into the design of vaccines is therefore considered advantageous.

Prostatic acid phosphatase (PAP) is a 386 amino acid protein secreted by the prostate. The expression of PAP is upregulated in prostate cancers, with increased circulating PAP levels being associated with advanced stage of disease and poor prognosis. PAP is highly prostate specific, and therefore represents a promising potential target antigen for cancer immunotherapy. Reverse immunology has been successfully used to identified immunogenic peptide HLA class-I-restricted epitopes as candidate target peptides for vaccine-based immunotherapy. Here we describe the identification of novel class-I, HLA-A2*0201, and class-II, HLA-DRB1*0101 and HLA-DRB1*0401 restricted peptides derived from human PAP, that are capable of stimulating in vivo CD8+ and CD4+ T-lymphocyte immune responses in HLA-transgenic mice.

These newly recognised peptides represent important, targets for T-lymphocyte based immunotherapy of this disease and have use as assays for cancer.

A first aspect of the invention provides a polypeptide comprising a sequence selected from:

(i)

SEQ. ID. 1

ILLWQPIPV (PAP.135),

(ii) a derivative sequence of the PAP.135 amino acid sequence having one or more amino acid deletions, additions, or substitutions, and

(iii) a fragment of the PAP.135 (i) or the derivative amino acid sequence (ii);

wherein the polypeptide has HLA class-I restricted activity.

A second aspect of the invention provides a polypeptide comprising a sequence selected from:

(i)

SEQ. ID. 2

CPRFQELESETLKSE (PAP.161),

(ii) a derivative sequence of the PAP.161 amino acid sequence having one or more amino acid deletions, additions or substitutions, and

(iii) a fragment of the PAP.161 (i) or the derivative amino acid sequence (ii);

wherein the polypeptide has an HLA class-II restricted activity.

The term polypeptide means 30 or less, preferably less than 25, 24, 23, 22, 21, 20, 19, 18, 17, 16, 15, 14, 13, 12, 11, 10, 9, 8, 7, 6 or 5 amino acid residues covalently joined to form the polypeptide.

Preferably 1, 2, 3, 4 or 5 amino acids are substituted, added or deleted. The production of such derivatives is achieved by methods known in the art. Preferably such derivatives have improved HLA class I or class II restricted activity. Amino acids are grouped into amino acids having similar properties, e.g.:
Hydrophobic: valine, leucine, isoleucine, methionine, proline
Aromatic: phenylalanine, tyrosine, tryptophan
Basic: lysine, arginine, histidine
Acidic: aspartate, glutamate
Amide: asparagine, glutamine
Nucleophilic: serine, threonine, cysteine
Small: glycine, alanine

Preferably, an amino acid of one group (e.g. basic amino acid) may be substituted for another amino acid from that group.

The “activity” of a peptide is a semi-quantitative measure of its immunogenic potency. For an MHC Class I-bound peptide, activity is preferably measured by the extent of lysis by cytotoxic T-cells of target cells displaying the MHC Class I peptide complexes. A peptide is usually considered to be immunogenic if it mediates killing of at least 15% of the cells that display it.

For MHC Class II-bound peptide, “activity” is usually a measure of the extent of T-cell proliferation induced by cells displaying the MHC Class I-peptide complexes.

More preferably the term “HLA class I activity” means that the polypeptide has activity selected from one or more of:

(i) HLA class I binding, such as to HLA-A2, especially to HLA-A2*0201. Preferably such binding is with high binding affinity,

(ii) Produces cytotoxicity in splenocytes derived from polypeptide immunised mice against (i) polypeptide (e.g. PAP.135), pulsed RMAS cells or (ii) prostate cancer cell-line cells, such as LNCaP cells. This cytotoxicity is preferably capable of being blocked with anti-HLA-A2 antibody, and/or

(iii) The polypeptide produces increase IFN-γ production in splenocytes from polypeptide immunised mice. This is compared with non-immunised mice.

More preferably the term “HLA class II activity” means the polypeptide has an activity selected from one or more of:

(i) HLA class II binding, such as to HLA-DR, such as HLA-DRβ1*0401 or HLA-DRβ1*0101, preferably with high binding specificity; and/or

(ii) Causes increased proliferation in T cells, for example by coincubating splenocytes from polypeptide immunised mice with polypeptide pulsed bone-derived dendritic cells. This is preferably blocked by using an anti-HLA-DR antibody. The mice used are preferably HLA-DRβ1*101 or HLA-DR1*401 mice.

Binding activity may be determined by techniques known in the art.

Preferably the methods of assaying such activity is as shown in the Materials and Methods section for PAP.135 or PAP.161 respectively.

In the algorithm used, the SYFPEITHY algorithm, peptides are compared with Influenza protein for both MHC Class I and MHC Class II. This is one of a number of different approaches to identifying such peptides, all of which have advantages and disadvantages. The use of this algorithm is merely a guide and does not mean that the peptides identified solely by this algorithm inevitably have desirable properties. A number of different peptides were identified as potentially having desirable properties. PAP.135 and PAT.161 were then identified as having the most desirable properties via further experimental work by the inventors.

These properties were not predictable from the algorithm. For example, they identified PAP.30, a peptide already known in the art (Peshwa, 1998), but indicated as having higher binding than PAP.135 using the algorithm. However, PAP.135 surprisingly was found to have better properties such as higher affinity to T2 cells when investigated further.

Preferably a further aspect of the invention provides a nucleic acid molecule selected from the group consisting of:

(a) Nucleic acid molecules encoding a polypeptide having the amino acid sequence depicted according to the invention; and

(b) Nucleic acid molecules, the complementary strand of which specifically hybridises to a nucleic acid molecule in (a).

The nucleic acid molecules of the invention may be DNA, cDNA or RNA. In RNA molecules “T” (Thymine) residues may be replaced by “U” (Uridine) residues.

The term “specifically hybridising” is intended to mean that the nucleic acid molecule can hybridise to nucleic acid molecules according to the invention under conditions of high stringency. Typical conditions for high stringency include 0.1×SET, 0.1% SDS at 68° C. for 20 minutes.

The nucleic acid molecules of the invention may be readily derived because the genetic code is well-known:

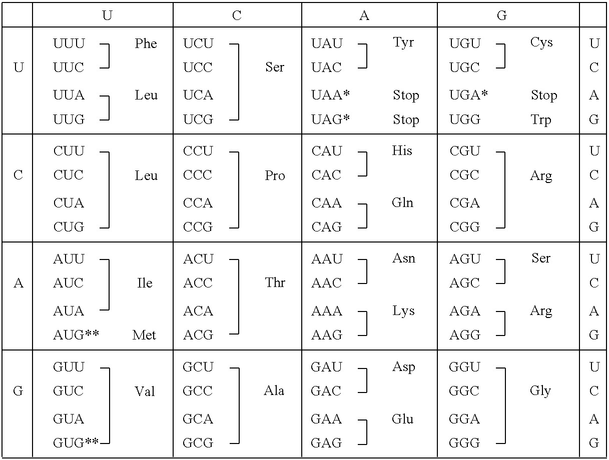

*Chain-terminating, or “nonsense” codons.
**Also used to specify the initiator formyl-Met-tRNAMet. The Val triplet GUG is therefore “ambiguous” in that it codes both valine and methionine.

The Genetic Code Showing mRNA Triplets and the Amino Acids for which they Code

The invention also includes within its scope vectors comprising a nucleic acid according to the invention. Such vectors include bacteriophages, phagemids, cosmids and plasmids. Preferably the vectors comprise suitable regulatory sequences, such as promoters and termination sequences which enable the nucleic acid to be expressed upon insertion into a suitable host. Accordingly, the invention also includes hosts comprising such a vector. Preferably the host is E. coli.

A second aspect of the invention provides an isolated polypeptide obtainable from a nucleic acid sequence according to the invention. As indicated above, the genetic code for translating a nucleic acid sequence into an amino acid sequence is well known.

Preferably the polypeptide comprises a sequence at least 80%, 85%, 90%, 95%, 96%, 97%, 98% or 99% identical to the PAP.135 or PAP.161 sequences. This can be determined conventionally using known computer programs such as the Bestfit program (Wisconsin Sequence Analysis Package, Version 8 for Unix, Genetics Computer Group, University Research Park, 575 Science Drive, Madison, Wis. 53711). When using Bestfit or any other sequence alignment program to determine whether a particular sequence is, for instance, 95% identical to a reference sequence according to the present invention, the parameters are set, of course, such that the percentage of identity is calculated over the full length of the reference amino acid sequence and that gaps in homology of up to 5% of the total number of amino acid residues in the reference sequence are allowed.

A further aspect of the invention provides the use of nucleic acids or polypeptides according to the invention, to detect or monitor cancers, preferably gastro-intestinal cancers, such as prostate cancer.

The use of a nucleic acid molecule hybridisable under high stringency conditions, a nucleic acid according to the third aspect of the invention to detect or monitor cancers, e.g. prostate cancer, is also encompassed. Such molecules may be used as probes, e.g. using PCR.

The expression of genes, and detection of their polypeptide products may be used to monitor disease progression during therapy or as a prognostic indicator of the initial disease status of the patient.

There are a number of techniques which may be used to detect the presence of a gene, including the use of Northern blot and reverse transcription polymerase chain reaction (RT-PCR) which may be used on tissue or whole blood samples to detect the presence of cancer associated genes. For polypeptide sequences in-situ staining techniques or enzyme linked ELISA assays or radio-immune assays may be used. RT-PCR based techniques would result in the amplification of messenger RNA of the gene of interest (Sambrook, Fritsch and Maniatis, Molecular Cloning, A Laboratory Manual, 2^{nd} Edition). ELISA based assays necessitate the use of antibodies raised against the protein or peptide sequence and may be used for the detection of antigen in tissue or serum samples (McIntyre C. A., Rees R. C. et al., Europ. J. Cancer 28, 58-631 (1990)). In-situ detection of antigen in tissue sections also rely on the use of antibodies, for example, immuno peroxidase staining or alkaline phosphatase staining (Gaepel, J. R., Rees, R. C. et.al., Brit. J. Cancer 64, 880-883 (1991)) to demonstrate expression. Similarly radio-immune assays may be developed whereby antibody conjugated to a radioactive isotope such as I¹²⁵ is used to detect antigen in the blood.

Blood or tissue samples may be assayed for elevated concentrations of the nucleic acid molecules or polypeptides.

The elevated polypeptide or nucleic acid may be PAP or nucleic acid molecules encoding PAP.

Preferably elevated levels of the molecules in tissues that are not normal prostate is indicative of the presence of cancerous tissues.

Methods of producing antibodies which are specific to the polypeptides of the invention, for example, by the method of Kohler & Milstein to produce monoclonal antibodies, are well known. A further aspect of the invention provides an antibody which specifically binds to a polypeptide.

Kits for detecting or monitoring prostate cancer using polypeptides, nucleic acids or antibodies according to the invention are also provided. Such kits may additionally contain instructions and reagents to carry out the detection or monitoring.

A further aspect of the invention provides for the use of nucleic acid molecules according to the third aspect of the invention or polypeptide molecules according to the first aspect of the invention in the prophylaxis or treatment of cancer, or pharmaceutically effective fragments thereof. By pharmaceutically effective fragment, the inventors mean a fragment of the molecule which still retains the ability to be a prophylactant or to treat cancer. The cancer may be prostate cancer.

The molecules are preferably administered in a pharmaceutically amount. Preferably the dose is between 1 μg/kg. to 10 mg/kg.

The nucleic acid molecules may be used to form DNA-based vaccines. From the published literature it is apparent that the development of protein, peptide and DNA based vaccines can promote anti-tumour immune responses. In pre-clinical studies, such vaccines effectively induce a delayed type hypersensitivity response (DTH), cytotoxic T-lymphocyte activity (CTL) effective in causing the destruction (death by lysis or apoptosis) of the cancer cell and the induction of protective or therapeutic immunity. In clinical trials peptide-based vaccines have been shown to promote these immune responses in patients and in some instances cause the regression of secondary malignant disease. Antigens expressed in prostate cancer (or other types of cancers) but not in normal tissue (or only weakly expressed in normal tissue compared to cancer tissue) will allow us to assess their efficacy in the treatment of cancer by immunotherapy. Polypeptides derived from the tumour antigen may be administered with or without immunological adjuvant to promote T-cell responses and induce prophylactic and therapeutic immunity. DNA-based vaccines preferably consist of part or all of the genetic sequence of the tumour antigen inserted into an appropriate expression vector which when injected (for example via the intramuscular, subcutaneous or intradermal route) cause the production of protein and subsequently activate the immune system. An example of DNA-based vaccine production is shown in, for example, Thompson S. A., et al. (J. Immunol. (1998), 160, pages 1717-1723.

An alternative approach to therapy is to use antigen presenting cells (for example, dendritic cells, DC's) either mixed with or pulsed with protein or peptides from the tumour antigen, or transfect DC's with the expression plasmid (preferably inserted into a viral vector which would infect cells and deliver the gene into the cell) allowing the expression of protein and the presentation of appropriate peptide sequences to T-lymphocytes.

Accordingly, the invention provides a nucleic acid molecule according to the invention in combination with a pharmaceutically-acceptable carrier.

A further aspect of the invention provides a method of prophylaxis or treatment of a cancer such as prostate cancer, comprising the administration to a patient of a nucleic acid molecule according to the invention.

The polypeptide may be attached to a protein or a fragment of a protein carrier, such as tetanus toxoid, which is not from PAP to make it immunogenic (using well-known techniques). Such constructs and nucleic acid molecules encoding such constructs are also part of the invention.

The polypeptide molecules according to the invention may be used to produce vaccines to vaccinate against prostate cancer.

Accordingly, the invention provides a polypeptide according to the invention in combination with a pharmaceutically acceptable carrier.

The invention further provides use of a polypeptide according to the invention in a prophylaxis or treatment of a cancer such as prostate cancer.

Methods of prophylaxis or treating a cancer, such as prostate cancer, by administering a polypeptide according to the invention to a patient, are also provided.

Vaccines comprising nucleic acid and/or polypeptides according to the invention are also provided.

The polypeptides of the invention may be used to raise antibodies. In order to produce antibodies to tumour-associated antigens procedures may be used to produce polyclonal antiserum (by injecting protein or peptide material into a suitable host) or monoclonal antibodies (raised using hybridoma technology). In addition PHAGE display antibodies may be produced, this offers an alternative procedure to conventional hybridoma methodology. Having raised antibodies which may be of value in detecting tumour antigen in tissues or cells isolated from tissue or blood, their usefulness as therapeutic reagents could be assessed. Antibodies identified for their specific reactivity with tumour antigen may be conjugated either to drugs or to radioisotopes. Upon injection it is anticipated that these antibodies localise at the site of tumour and promote the death of tumour cells through the release of drugs or the conversion of pro-drug to an active metabolite. Alternatively a lethal effect may be delivered by the use of antibodies conjugated to radioisotopes. In the detection of secondary/residual disease, antibody tagged with radioisotope could be used, allowing tumour to be localised and monitored during the course of therapy.

The term “antibody” includes intact molecules as well as fragments such as Fa, F(ab′)₂ and Fv.

The invention accordingly provides a method of treating a cancer such as prostate cancer, by the use of one or more antibodies raised against a polypeptide of the invention.

The cancer-associated proteins identified may form targets for therapy.

The invention also provides nucleic acid probes capable of binding sequences of the invention under high stringency conditions. These may have sequences complementary to the sequences of the invention and may be used to detect mutations identified by the inventors. Such probes may be labeled by techniques known in the art, e.g. with radioactive or fluorescent labels.

Preferably the cancer which is detected, assayed for, monitored, treated or targeted for prophylaxis, is prostate cancer.

Still further aspects of the invention provide polypeptides comprising a sequence selected from the amino acid sequence of PAP.284, PAP.15, PAP.64 or PAP.207, a derivative of the amino acid sequence having one or more substitutions, additions or deletions, or a fragment thereof, having HLA class I or HLA class II restricted activity.

Nucleic acid sequences, all of the uses of the polypeptides, methods of using such polypeptides, kits, and vaccines, as defined for the PAP.135 and PAP.161 sequences above, are also provided in the scope of the invention. That is, reference to the PAP.135 or PAP.161 amino acid sequences above, may be replaced by reference to the PAP.284, PAP.15, PAP.64 or PAP.207 amino acid sequence shown in Table I.

The invention will now be described by way of example only, with reference to the following figures:

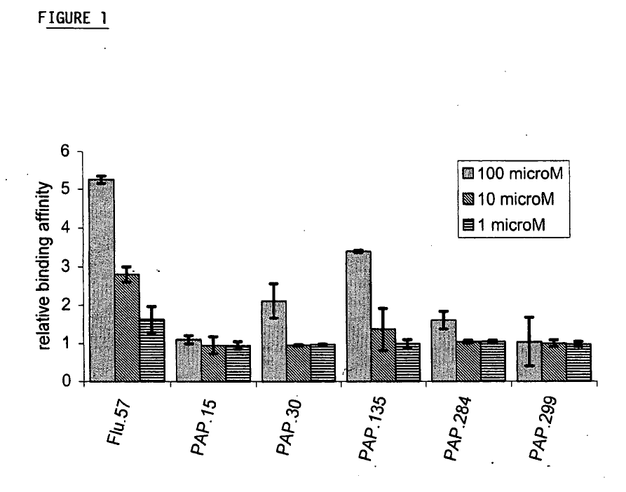

FIG. 1: HLA-A2 binding affinities of selected prostate acid phosphatase derived peptides

T2 cells incubated with peptides were stained for the presence of cell surface HLA-A2 antigen using flow cytometry. Fluorescent intensity of stained cells was used to determine the ability of test peptides to bind and therefore stabilise HLA-A2. Bars indicate the relative binding affinities (RBI) of the six peptides tested. The RBI was calculated for each peptide from the obtained mean fluorescence intensity (MFI) of T2 cells incubated with test peptide divided by the MFI obtained from cells incubated with 100% DMSO alone. Flu matrix peptide is included as a positive control. Error bars indicate 95% confidence intervals (t-test).

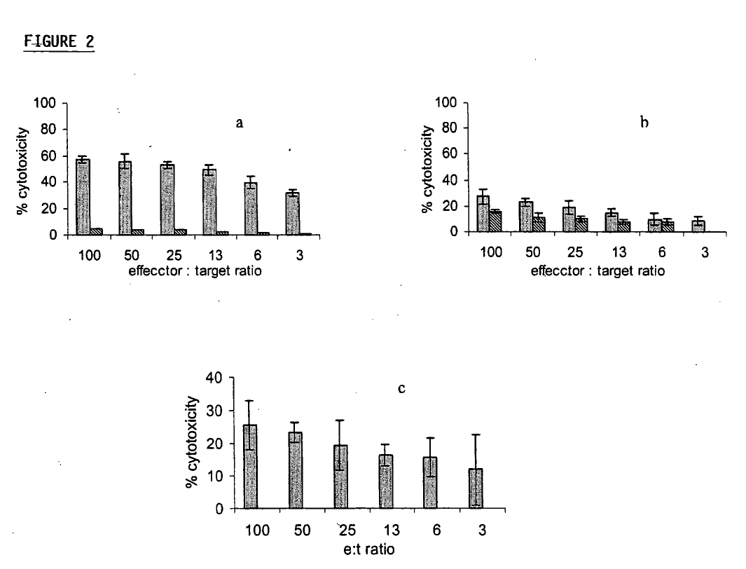

FIG. 2: Cytotoxicity of CTL derived from PAP.135 immunised mice against various target cells.

Cell lysis, expressed as a percentage of maximum cell lysis produced by incubation of cells in 0.5% SDS. Horizontal axis represents ratio of effector cells to target cells. Lysis of target cells pulsed with PAP.135 (dotted columns) control cells (pulsed with an irrelevant influenza HLA-A2 epitope) (diagonal striped columns). The results are representative of 5 separate experiments using 27 individual spleen cell preparations.

(a): Cytotoxicity against PAP.135 pulsed RMAS cells by splenocytes derived from PAP.135 immunised mice. Results are representative of 27 mice and 5 individual experiments

(b): Cytotoxicity against PAP.135 pulsed RMAS cells by splenocytes derived from non-immunised (naïve) mice. Results are representative of 5 mice and 5 individual experiments.

(c): Cytotoxicity against LNCaP cells by splenocytes derived from PAP.135 immunised mice. Results are representative of 10 mice and 2 individual experiments.

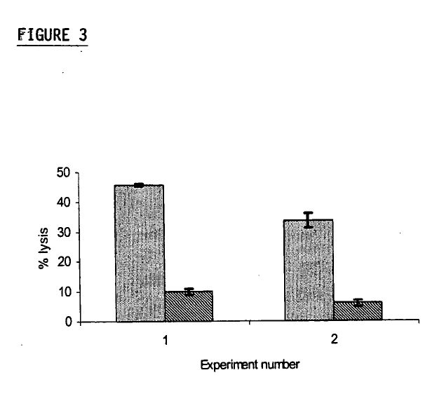

FIG. 3: Effect of anti-HLA-A2 antibody on peptide specific lysis of target cells by effector splenocytes

An inhibitory concentration of anti-HLA-A2 antibody was added to 2 out of 4 wells containing PAP.135-pulsed target cells and effector splenocytes derived from PAP.135 immunised mice. Columns indicate % lysis of targets in the absence (dotted) or presence (diagonal stripe) of blocking antibody. Addition of the HLA-A2 blocking antibody inhibited peptide specific lysis by approximately 80% in two separate experiments.

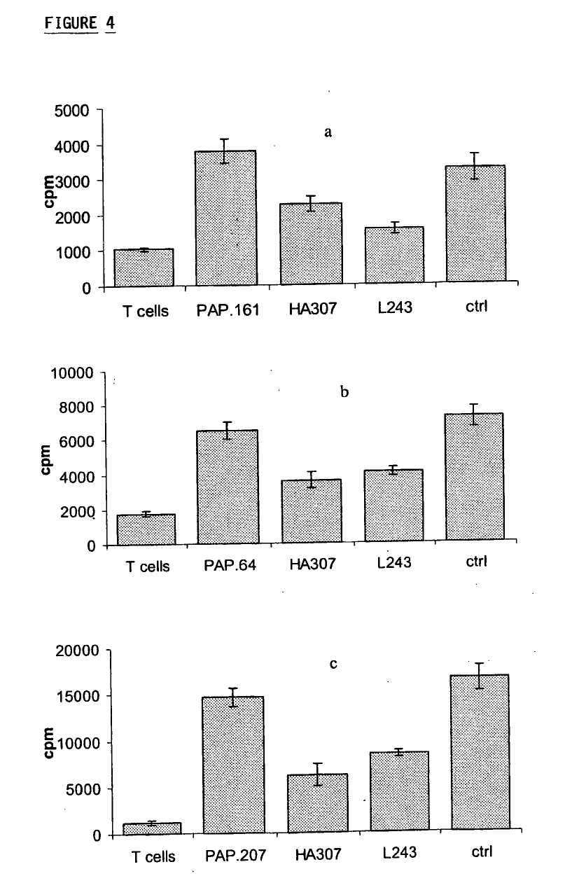

FIG. 4: Specific proliferation of splenocytes from immunised HLA-DR4 mice, re-stimulated in vitro with peptide in vitro for 5 days

Proliferation was inhibited by the addition of L243 antibody, an HLA-DR specific antibody but not isotype control. Coculture of splenocytes with mature DC pulsed with the Flu peptide induced a much reduced proliferation as shown by the limited incorporation of tritiated thymidine. (A: PAP.161, B: PAP.64 or C: PAP.207)

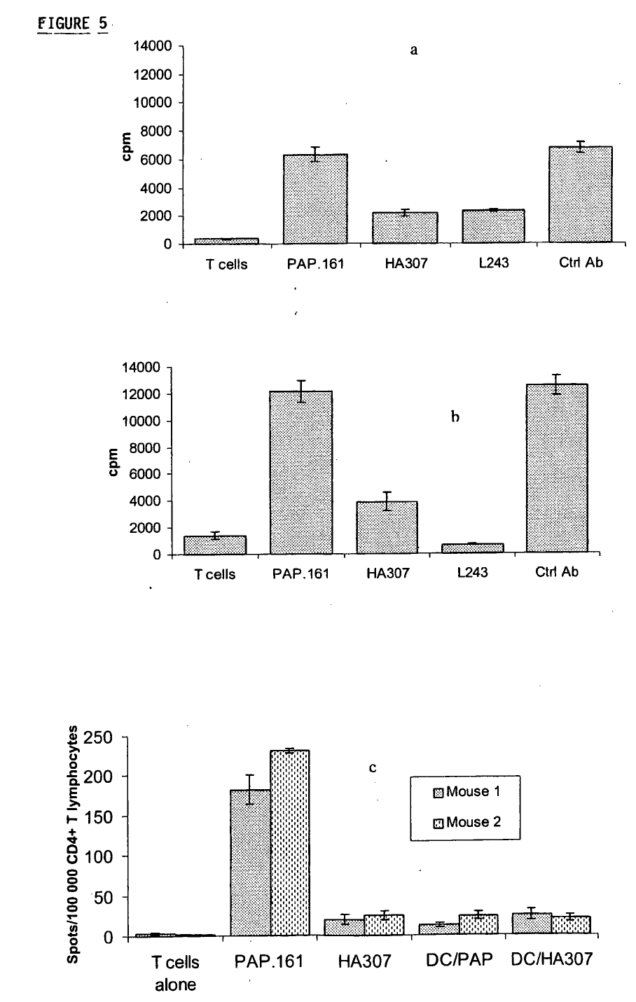

FIG. 5: Splenocytes from immunised animals were stimulated in vitro with the PAP.161 peptide

After 7 days in culture CD8+ T cells were removed and the cells were put back in culture for another 7 days. Thereafter cells were assessed for their proliferative response (Figure A mouse 1 and B mouse) or their production of IFN-γ ELISPOT assay to BM-DC pulsed with either the relevant peptide (PAP.161) or irrelevant peptide (Flu). As can be seen both mice responded specifically to DC pulsed with the PAP.161 peptide by proliferating (Figure A and B) and producing IFN-γ (Figure C). The proliferative response was DR restricted and was blocked by the presence of L243 antibody in the culture (Figure A and B) but unchanged by the presence of a control antibody.

### METHODS

Peptide Selection and Synthesis

Candidate peptides with either HLA-A2*0201 or HLA-DRB1*0401/HLA-DRB1*0101 binding motifs were identified using the SYFPEITHI on-line epitope prediction algorithm, which analyses peptides for the presence of certain amino acid residues which favour MHC binding. The peptide corresponding to positions 58-66 (GILGFVFT) of the influenza virus M1 protein has been previously identified as a potent HLA-A2*0201 CTL epitope and was employed as a positive control in CTL generation assays. For class-II proliferation assays, the influenza peptide corresponding to positions 307-319 of the influenza virus (PKYVKQNTLKLAT—SEQ. ID. 3) was used. The peptide corresponding to positions 128-140 (TPPAYRPPNAPIL—SEQ. ID. 4) of the hepatitis-B pre-core protein (AAK57285) is a known mouse MHC class-II epitope. All peptides were synthesized by Alta Bioscience (Birmingham, UK) and dissolved in 100% DMSO to a concentration of 10 mg/ml.

Mice

HLA-A2.1/K^{b} transgenic C57 black mice express the product of the HLA-A2.1/K^{b} chimeric gene in which the α-3 domain of the heavy chain is replaced by the H-2/K^{b} domain, but the HLA-A2.1 α-1 and α-2 domains are unaltered (N. Holmes, Cambridge University, UK). C57BL/6HLA-DR4 knockout/mice, were obtained from Taconic, USA and FVBN/DR1 mice were a generous gift from Altman D. M (MRC Clinical Sciences Centre, London). Mice were bred under license.

Other mice having the desired phenotype are also known in the art and could have been used instead. One such example are HHD II mice.

Cell-Lines

T2 and RMAS cells are lymphoblastoid cell-lines, which exhibit a deficiency in MHC class-I expression on the cell surface despite synthesizing normal HLA-A2 heavy chains and β₂-microglobulin. These cells lack the TAP1 and TAP2 genes located within the MHC class-II region of chromosome 6, which encode the Transport Associated Proteins (TAP) necessary for the transport of oligopeptides from the cytosol into the endoplasmic reticulum. T2 cells express human MHC class-I molecules, whereas RMAS cells express HLA-A2.1/K^{b} transgenic class-I, (RMAS-HLA-A2). This allows the murine CD8 molecule on murine CD8+ T-lymphocytes to interact with the syngeneic α-3 domain of hybrid MHC class-I molecule. Cells were grown in suspension in tissue culture flasks containing RPMI 1640 (GibcoBRL, UK) with 10% foetal calf serum (FCS) (BioWhittaker, Belgium) and 1% L-glutamine supplement (GibcoBRL, UK).

Again, alternative cell lines with the required phenotype which are known in the art, such as RMAS-A2 cells could also have been used.

LNCaP is a human prostate cancer cell-line known to express HLA-A2 and PAP. Cells were propagated in RPMI 1640 media (GibcoBRL, UK) supplemented with 10% foetal calf serum (BioWhittaker, Belgium), 100 U/ml penicillin with 100 μg/ml streptomycin (GibcoBRL, UK), 5 ng/ml hydrocortisone and 5 ng/ml testosterone (Sigma, USA). LCL-BM is a HLA-A2 positive human lymphoblastoid cell-line that does not express PAP. Cells were grown in suspension in tissue culture flasks containing RPMI 1640 (GibcoBRL, UK) with 10% foetal calf serum (FCS) (BioWhittaker, Belgium) and 1% L-glutamine supplement (GibcoBRL, UK).

T2-Binding Assay

T2 cells were washed twice in serum-free RPMI and incubated overnight at 37° C. in a round-bottomed 96 well plate in 50 μl serum-free RPMI containing 160,000 cells and final concentrations of 100, 10 and 1 μM of peptide. A negative control was provided by adding dilute DMSO without peptide to 2 wells. Peptide-induced stabilization of HLA-A2 molecules on the surface of T2 cells was measured by indirect immunofluorescence. After washing in phosphate buffered saline (PBS) (Oxoid, UK) containing 0.1% bovine serum albumin (BSA) (Sigma, USA), the cells were incubated on ice for 30 minutes with 20 μl of mouse antihuman HLA-A2 antibody MA2.1 (ATCC, UK). Following washing with PBS+BSA, 100 μl of a 1/100 dilution of FITC labelled goat anti-mouse IgG (Sigma, USA) was added and the cells incubated for 30 minutes on ice. The cells were washed twice using PBS+BSA, fixed in 500 μl Isoton and allowed to normalise to room temperature for 20 minutes prior to analysis by flow cytometry. The binding affinity was expressed as the ratio of mean channel of fluorescence observed of each peptide concentration to that of a control sample without peptide.

Immunisations

HLA-A2.1/K^{b} mice were immunised with an emulsion consisting of 100 μg of the putative class-I peptide epitope and 100 μg of non-specific class-II peptide derived from hepatitis B virus in 50% incomplete Freund's adjuvant (IFA). HLA-DR4- and HLA-DR1-transgenic mice were immunised twice at one week interval with 100 μg of class-II peptide emulsified in 50% incomplete Freund's adjuvant (IFA) and 50% PBS. All vaccine were delivered as a 100 μl intradermal bolus inoculum at the base of the tail.

Propagation of CTL in Vitro

After at least 7 and not more than 10 days, immunised animals were killed, and the spleens recovered into transport media. Spleens were gently macerated and flushed by injecting 10 ml “CTL culture media” (RPMI 1640, 1% L-glutamine, 10% FCS (PAA labs), 20 mM Hepes, 50 μM 2-mercaptoethanol, 50 U/ml penicillin, 500 U/ml streptomycin and 0.25 μg/ml fungizone) under pressure through a fine bore hypodermic needle introduced through the spleen capsule, thus facilitating the isolation of splenocytes. The remaining tissue was diced and digested for 1 hour at 37° C. using an enzyme cocktail (IMDM containing 50 mM 2-ME, 100 U/ml penicillin+1000 U/ml streptomycin, 8 mg/ml collagenase and 1% DNAse). The flushed and digested cell populations were washed and pooled, and then seeded into 15 wells of a 24 well-plate at 5×10⁶ cells/well in 2 ml of media containing 20 μg peptide. The test peptide was added to 12 wells, while 3 wells received an irrelevant class-I peptide as a control. Splenocyte cultures were incubated for 5 days at 37° C.

Cytotoxicity Assay

Target cells were pulsed overnight with test peptide or an irrelevant HLA-A2 binding epitope and labelled for 1 hour with 1.85 MBq ⁵¹Chromium. For cytotoxicity assays involving tumour cell-lines, cells were treated with 100 U/ml IFN-γ amma for 24 hours prior to ^{|}Chromium labelling. Target cells were washed and allowed to stand in CTL media for 1 hour before washing again. Target cells and effector CTLs were co-incubated at various ratios in 96-well plates in a final volume of 200 μl for 4 hours at 37° C. HLA-A2 dependence was determined by the addition of anti-HLA-A2 antibody (Serotec, UK) to some wells. Maximum and spontaneous release of ⁵¹Chromium was ascertained by incubating target cells with 2% SDS or media alone, respectively. Supernatants were harvested and analysed using a Top Count™ gamma counter (Canberra-Packard, USA). Specific lysis was determined as follows:

$$\% cell lysis = 100 \times \frac{[ experimental release ] - [ spontaneous release ]}{[ maximum release ] - spontaneous release ]}$$

Generation of Dendritic Cells from Bone-Marrow (BM-DC)

0DC were generated using a method adapted from Inaba et al (Inaba et al. 1992). Briefly, femurs and tibias were harvested aseptically from non-immunised DR1 or DR4 transgenic mice and placed in sterile PBS supplemented with 50 IU/ml penicillin, 50 μg/ml streptomycin, and 0.25 μg/ml fungizone. The marrow was flushed out of the bone using BM-DC media (RPMI 1640 medium supplemented with 2 mM glutamine, 5% FCS, 10 mM Hepes, 50 μM 2-ME, 50 μg/ml penicillin, 50 μg/ml streptomycin, 0.25 μg/ml fungisone and 10% of supernatant collected from X-63 cells). The cells were washed once in BM-DC media and plated in 24-well plates at 10⁶/well in 1 ml DC media and incubated at 37° C. in a 5% CO₂ humidified atmosphere. On days 2 and 4, the non-adherent cells (T cells, B cells, granulocytes) were removed, and the remaining cells were cultured in fresh DC medium. After 7-9 days, clusters of loosely adherent DC were dislodged and collected by gently pipetting, before being washed and seeded in 24-well plates at 0.5×10⁶ cells/well in 1 ml DC media. Peptides were added at 1 μg/well and the plates were incubated at 37° C. 1 μg/ml of LPS was added after 5 hours, and the plates were incubated overnight at 37° C. in a 5% CO₂ in air-humidified atmosphere.

Restimulation of T Helper Cells in Vitro

After 6 days, the spleens from immunised animals were collected and the cells flushed out using T cell media (RPMI+10% FCS+20 mM HEPES buffer+50 μM 2-mercaptoethanol+50 U/ml penicillin/streptomycin+0.25 μg/ml fungizone). The remaining splenic tissue was digested using an enzyme cocktail (0.1 U/ml DNAase (Sigma)+1.6 mg/ml collagenase (Sigma)) for 1 hour at 37° C. The cell suspension obtained was pooled with the flushed cells, washed once, and the splenocytes were plated in 24 well plates with 10 μg/ml of peptide at a density of 2.5×10⁶/ml for FVB/N-DR1 or 3.5×10⁶/ml for C57BL/6-DR4. Cells were also cultured with an irrelevant peptide as a control. To measure cytokine production, culture supernatants were collected on day 2 and 5 of the culture for the measurement of cytokine by ELISA.

Proliferative Responses of T-cell to Potentially Immunogenic Peptides

Splenocytes from immunised animals cultured for 6 days in the presence of 10 μg of the test peptide were harvested, washed and counted. CD8+ T cells were depleted from the cell suspension using anti-CD8+ antibody coupled with magnetic beads (Dynal, UK) as per the manufacturers instructions. Cells were then washed in PBS and resuspended at 5×10⁵/ml in T cell medium. 100 μl of T cells was then added to all wells. Anti-DR antibody (L243) or isotype control were added to some wells to confirm the MHC specificity of the T-cell response. LPS-treated dendritic cells were harvested, washed and incubated in 1 ml of DC medium with the addition of 10 μg of peptide at 37° C. for 5 hours. After washing, 103 peptide-pulsed DC were added to the relevant wells of lymphocyte cultures in 50 μl media. These plates were then incubated for 3 days at 37° C. in a 5% CO₂ in air-humidified atmosphere. Tritriated thymidine was then added to the culture and incubated again for 18 hours. Plates were harvested onto 96 Uni/Filter (Packard), the scintillation liquid (Microscint 0, Packard) was added and the plates were counted on a Top-Count counter (Packard). Results are expressed in counts per minute (cpm) and as a means of the triplicate wells. Statistical analysis was performed using unpaired Student's t test.

IFN-γELISPOT and Capture of Peptide-Reactive CD4+ T Cells

Mature DC were harvested and pooled on day 9, and incubated with 10 ug of PAP.161 or HA307 peptide as a negative control. Cells were incubated for 4-5 hours before being seeded onto a Nitrocellulose-backed 96 well plates (Millipore) at 1×10⁵ cells/well. Before the addition of cells plates had been coated overnight at 4° C. with anti-IFN-γ antibody (R&D system, UK) as per manufactures recommendations. Rested CD8⁻ T cells were harvested counted and added to the plate at 1×10⁵ cells/well using T cells media. After 24 hrs at 37° C. in a water-saturated atmosphere, the plates were washed extensively with a solution of 0.05% Tween 20/PBS and supplemented with the biotinylated ant-IFN-γ detection antibody (R&D system, UK). After incubation for 2 hrs at 37° C., plates were washed and developed with ELIspot development module (R&D system). Controls were the effector cells alone (spontaneous IFN-γ release), the APCs alone and the effector co-culture with DC pulsed with the irrelevant peptide (HA307 peptide).

Cytokine Production by CTL Culture

Aliquots of supernatants were removed from CTL cultures on days 1 and 5 and on days 2 and 5 from CD4⁺ T cell cultures and stored at −20° C. until required. ELISA was used to quantitate concentrations of murine IFN-γ and IL-5 (R&D systems, UK).

Results

Peptide Prediction and Binding Affinity of Class-I Peptides

The SYFPEITHI on-line epitope prediction algorithm was used to identify motifs derived from human PAP that comprise amino acid residues favoring HLA-A2*0201, HLADRB1*0101 or HLA-DRB1*0401 binding. Table 1 illustrates those peptides selected for further analysis. As well as novel peptides not previously reported, two class-I PAP peptides were included that had previously been shown to be strong HLA-A2 binders (Table 1).

TABLE 1

| | Amino-acid | | |
|---|---|---|---|
| Seq. ID. No. | sequence | Name | Description |
| SEQ. ID. 5 | ALDVYNGLL | PAP.299 | |
| SEQ. ID. 6 | VLAKELKFV | PAP.30 | |
| SEQ. ID. 7 | IMYSAHDTTV | PAP.284 | novel |
| | | | (predicted) |
| | | | epitope |
| SEQ. ID. 8 | ILLWQPIPV | PAP.135 | novel |
| | | | (predicted) |
| | | | epitope |
| SEQ. ID. 9 | ALASCFCFFC | PAP.15 | novel |
| | | | (predicted) |
| | | | epitope |
| SEQ. ID. 10 | PQGFGQLTQLGMEQH | PAP.64 | novel |
| | | | (predicted) |
| | | | epitope |
| SEQ. ID. 11 | CPRFQELESETLKSE | PAP.161 | novel |
| | | | (predicted) |
| | | | epitope |
| SEQ. ID. 12 | SKVYDPLYSESVHNF | PAP.207 | novel |
| | | | (predicted) |
| | | | epitope |

Table 1: Predicted Peptides HLA Class-I and Class-II-Restricted

Candidate HLA-A2*0201, HLA-DRB1*0101 and HLA-DRB1*0401 specific epitopes derived from human PAP, based on predicted binding affinities using the SYFPEITHI algorithm.

To determine the binding ability of predicted peptides to HLA-A2*0201 antigen, an in vitro cellular binding assay was performed using the TAP-deficient cell-line T2. The peptide corresponding to positions 58-66 (GILGFVFT—SEQ. ID. 13) of the influenza virus M1 protein was used as a positive control. The Fluorescence Indices (FI) for all peptides at 100 μM, 10 μM and 1 μM concentrations are shown in FIG. 1 and all peptides were tested in three independent experiments. PAP.299 and PAP.15 peptides showed no significant binding to HLA-A2. Weak binding was exhibited by PAP.284, while PAP.30 and particularly PAP.135 showed strong binding to HLA-A2, in comparison with the positive control influenza peptide.

Generation of CTL Activity to PAP-Derived Class-I Peptide

PAP.135 was tested for its ability to stimulate a peptide-specific CTL mediated immune response in vivo. HLA-A2.1/K^{b} transgenic mice were immunised with a peptide emulsion, and 7-10 days later spleen cells from these mice were restimulated with peptide in vitro and subsequently tested for HLA-A2-restricted, peptide-specific CTL activity. Splenocytes from immunised mice lysed RMAS-HLA-A2 cells, pulsed with PAP.135 in a dose dependent manner but not in control target cells (FIG. 2-a). Peptide specificity was evidenced by the ability of splenocytes to lyse cells pulsed with PAP.135, but not an irrelevant HLA-A2*0201 CTL epitope. Splenocytes derived from non-immunised (naïve) mice and stimulated by PAP.135 in vitro were also tested but failed to demonstrate cytotoxicity to PAP.135 pulsed RMAS-HLA-A2 cells (FIG. 2-b). The ability of splenocytes from HLA-A2.1K^{b} transgenic mice immunised with PAP.135 to recognise endogenously synthesised and processed PAP-HLA-restricted epitopes was investigated using the human prostate cancer cell-line, LNCaP, which express both HLA-A2 and PAP. FIG. 2-c illustrates the dose-dependent lysis of LNCaP cells, whereas LCL cells, which are known to express HLA-A2 but not PAP, were not susceptible to lysis. To confirm that the observed lysis of targets was mediated though the action of CD8+ CTLs, the ability of an anti-HLA-A2 antibody to block cytotoxicity was tested. The addition of HLA-A2 blocking antibody inhibited the lysis of target cells by approximately 80%, as shown in FIG. 3.

IFN-γ Production by T-Lymphocytes Responding to PAP.135 was Measured by ELISA IFN-γ production was low on day 0 of culture, but rose significantly by day 5 and splenocytes stimulated with PAP.135 in vitro released significantly more IFN-γ than those incubated with an irrelevant epitope (Table 2). The amount of IFN-γ released by splenocytes derived from non-immunised (naïve) mice was independent of the peptide used for in vitro stimulation (i.e. PAP.135 or the irrelevant peptide).

TABLE 2

| | | |
|---|---|---|
| | % cytoxicity at 50:1 ratio | |
| Exp. | RMAS + PAP.135 | IFN-γ release |
| No. | RMAS + irrel. | (PAP.135:irrel.) |

| | | | |
|---|---|---|---|
| 1 | 25 | 3 | 3 |
| 2 | 57 | 1 | 3.1 |
| 3 | 68 | 2 | 3.4 |
| 4 | 30 | 7 | 4.3 |
| 5 | 69 | 7 | 4.9 |
| 6 | 65 | 9 | 15 |
| 7 | 56 | 4 | 6 |
| 8 | 66 | 1 | 2.5 |
| 9 | 63 | 4 | 2.1 |
| 10 | 65 | 5 | 2.6 |
| 11* | 13 | 11 | 1.1 |

*Naïve mouse

Table 2: Interferon-Gamma Release by Splenocyte Cultures during in vitro Stimulation with PAP.135 or an Irrelevant Peptide

Figures represent the ratio of IFN-γ released by splenocytes stimulated in vitro by PAP.135 to IFN-γ released by splenocytes stimulated in vitro by an irrelevant HLA-A2*0201 CTL epitope. The mean results of 10 experiments using splenocytes derived from PAP.135-immunised mice are given, and 1 in which splenocytes derived from non-immunised (naive mice) were used.

HLA-DR Class-II Restricted Responses to PAP Peptides

In order to assess the immunogenicity of predicted MHC class-II peptides derived from the PAP protein, HLA-DRβ1*0401 and HLA-DRβ1*0101 transgenic mice were immunised twice with peptide emulsified in IFA and splenocytes from immunised mice were re-stimulated in vitro with the relevant peptide for 5 days. Bone-marrow-derived dendritic cells were generated concurrently and matured with LPS in the presence of peptide. Specific proliferation of T-cells was tested by co-incubating splenocytes and mature, peptide-pulsed DC in the presence or absence of anti-HLA-DR antibody. FIG. 4 illustrates the typical proliferation responses by HLA-DRβ1*0401 mice using peptide PAP.161 (FIG. 4-a), PAP.64 (FIG. 4-b) or PAP.207 (FIG. 4-c). Proliferation was also observed using HLA-DRβ1*0101 transgenic mice, however the response obtained was less potent than those observed with HLA-DRβ1*0401 transgenic mouse splenocytes. The response rates to PAP class-II peptides are summarized in table 3. Splenocytes from mice immunised with PAP.161 peptide were stimulated in vitro for 7 days in the presence of the peptide, thereafter the CD8+ T cells were removed and the cells “rested” in culture for 7 days before the addition of peptide-pulsed BM-DC. As shown in FIGS. 5a and 5b. Significant proliferation was obtained in immunised mice; furthermore, this proliferation was blocked in the presence of HLA-DR antibody but not with the isotype control antibody. The same cells were used in an ELISPOT assay and were shown to produce high amount of IFN-γ in peptide specific manner (FIG. 5c).

TABLE 3

| PEPTIDE | HLA-DR1 | HLA-DR4 |
|---|---|---|
| PAP.161 | 3/6 | 4/6 |
| PAP.64 | 1/6 | 2/5 |
| PAP.207 | 0/5 | 1/6 |

Table 3: HLA-DR-Restricted Peptide Stimulation

Total number of HLA-DR1 and HLA-DR4 mice whose splenocytes demonstrated specific proliferation after re-stimulation in vitro with relevant peptide.

Discussion

The limited treatment options for patients with hormone refractory prostate cancer has stimulated interest in the development of alternative therapies, including immunotherapy. The activation of effector T-cells capable of recognising and destroying prostate cancer cells can be achieved through various mechanisms, that can be applied at all stages of the disease. Reported clinical studies employing whole-cell vaccines in prostate cancer patients have to date met with only limited success, possibly because of the simultaneous presentation of large numbers of unselected antigenic determinants to the immune system. Immune targeting using a limited number of epitopes, specifically selected for their ability to induce CTL-mediated tumour lysis, represents an additional immunotherapeutic approach; vaccines based on either APC pulsed with MHC class I peptides or peptides alone have been investigated with promising preliminary results. However, to date few MHC class I and class II peptides derived from prostate specific proteins have been identified.

Animal studies have indicated the potential for PAP as for immunotherapy target and PAP derived peptides have been demonstrated to induce antigen-specific CTL responses in human studies. Human T-helper cell response has been shown in vitro, although the precise HLA haplotype to which the CD4+ T cells proliferated was not clearly defined. The data presented here using HLA-transgenic mice, identifies new HLA class-I and class-II-restricted PAP peptides that represent candidates for targeted immunotherapy.

The immunogenicity of CTL epitopes largely correlates with their ability to bind MHC molecules, where the co-incubation of putative class-I epitopes with TAP-deficient T2 cells allowed the selection of strong HLA-A2 binding peptides for further study. The binding affinity of PAP.30 has been described previously, and moderate HLA-A2 binding of this peptide was confirmed in this study; however, we were unable to confirm the strong HLA-A2 binding described for PAP.299. The strongest HLA-A2 binding was shown by PAP.135, a peptide not previously investigated for class-I binding, which showed a significantly higher binding affinity than either PAP.30 or PAP.299 peptides.

HLA-A2*0201 is the most common HLA class-I phenotype, and therefore peptides derived from cancer specific antigens that are able to stimulate a CTL reaction though presentation by HLA-A2*0201 represent potentially useful therapeutic agents in cancer immunotherapy. HLA-A2.1/Kb transgenic mice have been used successfully to identify HLA-A2*0201 restricted CTL epitopes. Peptide immunisation using PAP.135 in combination with a generic MHC class-II helper epitope derived from hepatitis B virus in IFA induced peptide specific HLA-A2.1 restricted CTL. CTL cultures derived from PAP.135 were highly cytotoxic towards target cells pulsed with peptide and human LNCaP cells expressing HLA-A2*0201 and PAP antigen (Hroszewicz et al 1983). Furthermore, the lysis of target cells by PAP.135 CTLs was shown to be both peptide specific and mediated through HLA-A2 presentation; splenocyte cultures derived from non-immunised (naïve) mice were unable to lyse target cells. IFN-γ release by splenocytes derived from PAP.135-immunised mice was significantly upregulated in the presence of PAP.135 in vitro.

We further investigated HLA-DR class-II restricted responses to PAP peptides that were predicted to bind to HLA-DR1 and HLA-DR4 according to the evidence-based computer assisted algorithm SYFPEITHI. The peptides displaying high binding scores for both HLA-DR alleles were selected and further studied (Table 1). C57BL/6-DR4 mice were immunised with these predicted HLA-DR-restricted PAP and the proliferative and IFN-γ response to peptide stimulation in vitro was monitored by proliferation assays and cytokine measurements. PAP.64 and PAP.207 peptide induced proliferative responses in 2 out of 5 and in 1 out of 6 immunised mice respectively (FIGS. 4B and 4C); however IFN-γ was not produced specifically to these peptide. These proliferative responses were blocked in the presence of L243 antibody, confirming the HLA-DR restriction of the observed response. The CD8-depleted splenocytes of 6 out of 8 mice immunised with PAP.161 peptide showed peptide specific proliferation, which was blocked by the addition of L243 antibody in the cultures (FIG. 4A). Moreover, IFN-γ was produced in large quantities by splenocytes cultured with the relevant peptide, but not with an irrelevant peptide. The immunogenicity of PAP.161 peptide was consistently confirmed when the CD8-depleted splenocytes were rested for 7 days and the response was assessed by proliferation assays (FIG. 5). These data indicated that PAP.161 peptide is immunogenic in an HLA-DR4-restricted manner in C57BL/6-DR4 mice.

Using FVB/N-DR1 mice PAP.64 and PAP.207 peptides failed, with the exception of one out six experiments, to elicit a proliferative responses or induce IFN-γ or IL-50 production of splenocytes (Table 3). CD8-depleted splenocytes proliferated in response to the PAP.161 in 3 out of 6 immunised mice, however no production of cytokines could be detected in high enough levels (Data not shown), suggesting that PAP.161 is promiscuous for HLA-DR1.

Collectively, these data demonstrate that ability of the PAP.135 epitope to stimulate HLA-A2 specific CTL activity in vivo and confirming the predicted immunogenicity of the class-II-restricted PAP.161 peptide in a HLA-DR1 and HLA-DR4 transgenic mice. These studies allow us to propose PAP.135 and PAP.161 as HLA class-I and class-II peptide targets, which are likely to have therapeutic, prophylatic and assay uses.

### REFERENCES

Bednarek M A, Sauma S Y, Gammon M C, Porter G, Tamhankar S, Williamson A R and Zweerink H J (1991). “The minimum peptide epitope from the influenza virus matrix protein. Extra and intracellular loading of HLA-A2”. Journal of Immunology, 147(12): 4047-53.
Disis M L, Gralow J R, Bernhard H, Hand S L, Rubin W M and Cheever M A (1996). “Peptide-based, but not whole protein, vaccines elicit immunity to HER-2/neu, oncogenic self-protein”. Journal of Immunology, 156(9): 3151-8.
Drijfhout J, Brandt R, D'Amaro J, Kast W and Melief C. (1995). “Detailed motifs for peptide binding to HLA-A*0201 derived from large random sets of peptides using a cellular binding assay”. Human Immunology 43: 1-12.
Eaton J, Perry M, Nicholson S, Guckian M, Russell N, Whelan M and Kirby R (2002). “Allogeneic whole-cell vaccine: a phase I/II study in men with hormone-refractory prostate cancer”. BJU International 89(1): 19-26.
Fong L, Ruegg C L, Brockstedt D, Engleman E G and Laus R (1997). “Induction of tissue-specific autoimmune prostatitis with prostatic acid phosphatase immunization: implications for immunotherapy of prostate cancer”. J. Immunol. 159(7): 3113-7.
Heukamp L C, Van der Burg S H, Drijfhout J W, Melief C J, Taylor-Papadimitriou J and Offringa R (2001). “Identification of three non-VNTR MUC1-derived HLA-A*0201-restricted T-cell epitopes that induce protective anti-tumor immunity in HLA-A2/K(b)-transgenic mice”. International Journal of Cancer 91(3): 385-92.
Horoszewicz, J S, Leong, S S, Kawinski, E, Karr, J P, Rosenthal, H, Chu, T M, Mirand, E A and Murphy, G P (1983). “LNCaP model of human prostatic carcinoma”. Cancer Research 43(4): 1909-18.
Hrouda D, Todryk S, Perry M, Souberbielle B, Kayaga J, Kirby R and Dalgleish A (2000). “Allogeneic whole-tumour cell vaccination in the rat model of prostate cancer”. BJU International 86(6): 742-748.
Inaba K, Inaba M, Romani N, Aya H, Deguchi M, Ikehara S, Muramatsu S, Steinman R M (1992). “Generation of large numbers of dendritic cells from mouse bone marrow cultures supplemented with granulocyte/macrophage colony-stimulating factor”. J. Exp. Med. 176: 1693.
Jacobs E L and Haskell C M (1991). “Clinical use of tumor markers in oncology”. Curr. Probl. Cancer 15(6): 299-360.
Kawashima I, Hudson S, Tsai V, Southwood S, Takesako K, Appella E, Sette A and Celis E (1998). “The multi-epitope approach for immunotherapy for cancer: identification of several CTL epitopes from various tumor-associated antigens expressed on solid epithelial tumors”. Human Immunology 59(1): 1-14.
Kim C J, Parkinson D R and Marincola F (1998). “Immunodominance across HLA polymorphism: implications for cancer immunotherapy”. Journal of Immunotherapy 21(1): 1-16.
Lam K W, Li C Y, Yam L T, Sun T, Lee G and Ziesmer S (1989). “Improved immunohistochemical detection of prostatic acid phosphatase by a monoclonal antibody”. Prostate 15(1): 13-21.
McNeel D, Nguyen L and Disis M (2001). “Identification of T helper epitopes from prostatic acid phosphatase”. Cancer Research 61(13): 5161-5167.
Meidenbauer N, Harris D, Spitler L and Whiteside T (2000). “Generation of PSA-reactive effector cells after vaccination with a PSA-based vaccine in patients with prostate cancer”. Prostate 43(2): 88-100.
Moss D J, Schmidt C, Elliott S, Suhrbier A, Burrows S and Khanna R (1996). “Strategies involved in developing an effective vaccine for EBV-associated diseases”. Advances in Cancer Research 69: 213-45.
Murphy G, Tjoa B, Simmons S, Jarisch J, Bowes V, Ragde H, Rogers M, Elgamal A, Kenny G, Cobb O, Ireton R, Troychak M, Salgaller M and Boynton A (1999a). “Infusion of dendritic cells pulsed with HLA-A2-specific prostate-specific membrane antigen peptides: a phase II prostate cancer vaccine trial involving patients with hormone-refractory metastatic disease”. Prostate 38(1): 73-78.
Murphy G, Tjoa B, Simmons S, Ragde H, Rogers M, Elgamal A, Kenny G, Troychak M, Salgaller M and Boynton A (1999b). “Phase II prostate cancer vaccine trial: report of a study involving 37 patients with disease recurrence following primary treatment”. The Prostate 39: 54-59.
Peshwa M, Shi J, Ruegg C, Laus R and Schooten W v (1998). “Induction of prostate tumor-specific CD8+ cytotoxic T-lynphocytes in vitro using antigen-presenting cells pulsed with prostatic acid phosphatase peptide”. Prostate 36(2): 129-138.
Quinn M and Babb P (2002a). “Patterns and trends in prostate cancer incidence, survival, prevalence and mortality. Part I: international comparison. [Review][99 refs]”. BJU International 90(2): 162-73.
Quinn M and Babb P (2002b). “Patterns and trends in prostate cancer incidence, survival, prevalence and mortality. Part II: individual countries. [Review][51 refs]”. BJU International 90(2): 174-84.
Rammensee H, Bachmann J, Emmerich N P, Bachor O A and Stevanovic S (1999). “SYFPEITHI: database for MHC ligands and peptide motifs”. Immunogenetics 50(3-4): 213-9.
Rammensee H G, Falk K and Roszschke O (1993). “Peptides naturally presented by MHC class I molecules”. Annu. Rev. Immunol. 11: 213-44.
Ruppert J, Sidney J, Celis E, Kubo R T, Grey H M and Sette A (1993). “Prominent role of secondary anchor residues in peptide binding to HLA-A2.1 molecules”. Cell 74(5): 929-37.
Saffran D, Reiter R, Jakobovits A and Witte O (1999). “Target antigens for prostate cancer immunotherapy”. Cancer Metastasis Rev 18(4): 437-449.
Salter R D and Cresswell P (1986). “Impaired assembly and transport of HLA-A and -B antigens in a mutant TxB cell hybrid”. Embo J 5(5): 943-9.
Salter R D, Howell D N and Cresswell P (1985). “Genes regulating HLA class I antigen expression in T-B lymphoblast hybrids”. Immunogenetics 21(3): 235-46.
Sette A, Vitiello A, Reherman B, Fowler P, Nayersina R, Kast W M, Melief C J, Oseroff C, Yuan L, Ruppert J and et al. (1994). “The relationship between class I binding affinity and immunogenicity of potential cytotoxic T cell epitopes”. Journal of Immunology 153(12): 5586-92.
Simons J, Mikhak B, Chang J, DeMarso A, CArducci M, Lim M, Weber C, Baccala A, Goemann M, Clift S, Ando D, Levitsky H, Cohen L, Sanda M, Mulligan R, Partin A, Carter H, Piantadosi S, Marshall F and Nelson W (1999). “Induction of immunity to prostate cancer antigens: results of a clinical trial of vaccination with irradiated autologous prostate tumour cells engineered to secrete granulocyte-macrophage colony stimulating factor using ex-vivo gene transfer”. Cancer Research 59(20): 5160-5168.
Sinha A A, Quast B J, Wilson M J, Reddy P K, Fernandes E T, Ewing S L and Gleason D F (1998). “Immunocytochemical localization of an immunoconjugate (antibody IgG against prostatic acid phosphatase conjugated to 5-fluoro-2′-deoxyuridine) in human prostate tumors”. Anticancer Res. 18(3A): 1385-92.
Small E, Fratesi P, Reese D, Strang G, Laus R, Peshwa M and Valone F (2000). “Immunotherapy of hormone-refractory prostate cancer with antigen-loaded dendritic cells”. Journal of Clinical Oncology 18(23): 3894-3903.
Solin T, Kontturi M, Pohlmann R and Vihko P (1990). “Gene expression and prostate specificity of human prostatic acid phosphatase (PAP): evaluation by RNA blot analyses”. Biochem Biophys Acta 1048(1): 72-7.
Theobald M, Biggs J, Dittmer D, Levine A J and Sherman L A (1995). “Targeting p53 as a general tumor antigen”. Proc. Natl. Acad. Sci. USA 92(26): 11993-7.
Thomson S, Elliot S, Sherritt M, Sproat K, Coupar B, Scalzo A, Forbes C, Ladhams A, Mo X, Tripp R, Doherty P, Moss D and Suhrbier A (1996). “Recombinant polyepitope vaccines for the delivery of multiple CD8 cytotoxic T cell epitopes”. Journal of Immunology 157: 822-826.
Vitiello A, Marchesini D, Furze J, Sherman L A and Chesnut R W (1991). “Analysis of the HLA-restricted influenza-specific cytotoxic T lymphocyte response in transgenic mice carrying a chimeric human-mouse class I major histocompatability complex”. Journal of Experimental Medicine 173(4): 1007-15.

1.-24. (canceled)

25. A polypeptide comprising a sequence selected from the group consisting of:
(a) ILLWQPIPV (SEQ ID NO: 1),
(b) a derivative sequence of SEQ ID NO: 1, said derivative sequence having one or more amino acid deletions, additions, or substitutions,
(c)
(SEQ ID NO: 2)
CPRLFQELESETLKSE,
(d) a derivative sequence of SEQ ID NO: 2, said derivative sequence having one or more amino acid deletions, additions or substitutions, and
(e) a fragment sequence of sequence (a), (b), (c), or (d);
wherein the polypeptide has HLA class-I restricted activity.

26. An isolated nucleic acid molecule comprising a sequence selected from the group consisting of:
(a) a nucleic acid molecule encoding the polypeptide of claim 25; and
(b) a nucleic acid molecule, the complementary strand of which specifically hybridises to a nucleic acid molecule encoding the polypeptide of claim 25.

27. A vector comprising a nucleic acid molecule according to claim 26.

28. A host cell comprising a vector according to claim 27.

29. A monoclonal antibody capable of specifically binding to the polypeptide of claim 25.

30. A method of detecting or monitoring cancer in a patient, the method comprising the step of detecting or monitoring elevated levels of the nucleic acid molecule according to claim 26 in a sample from the patient.

31. A method of detecting or monitoring cancer in a patient, the method comprising the step of detecting or monitoring elevated levels of the nucleic acid molecule according to claim 26 with another nucleic acid molecule or a probe in combination with a reverse transcription polymerase chain reaction.

32. A method of detecting or monitoring cancer in a patient, the method comprising the step of detecting or monitoring elevated levels of the polypeptide according to claim 25.

33. The method according to claim 32 wherein the detecting or monitoring step includes an antibody selective for the polypeptide of claim 25 to detect the polypeptide.

34. The method according to claim 33 further comprising the step of using an enzyme-linked immunosorbant assay.

35. The method according to claim 30 wherein the cancer is a prostate cancer.

36. A method of prophylaxis or treatment of cancer in a patient, the method comprising the step of administering to the patient a pharmaceutically effective amount of the nucleic acid molecule according to claim 26, or a pharmaceutically effective fragment thereof.

37. A method of prophylaxis or treatment of cancer in a patient, the method comprising the step of administering to the patient a pharmaceutically effective amount of a nucleic acid molecule hybridisable under high stringency conditions to the nucleic acid molecule according to claim 26, or a pharmaceutically effective fragment thereof.

38. A method of prophylaxis or treatment of cancer in a patient, the method comprising the step of administering to the patient a pharmaceutically effective amount of a polypeptide according to claim 25, or a pharmaceutically effective fragment thereof.

39. A method of prophylaxis or treatment of cancer in a patient, the method comprising the step of administering to the patient a pharmaceutically effective amount of the monoclonal antibody according to claim 29.

40. The method of prophylaxis or treatment of cancer according to claim 36, wherein the cancer is a prostate cancer.

41. A polypeptide comprising a protein carrier, which is not PAP or another fragment of PAP, covalently attached to the polypeptide according to claim 25, or a pharmaceutically effective fragment thereof.

42. A nucleic acid molecule encoding a polypeptide according to claim 41.

43. A vaccine comprising the nucleic acid molecule according to claim 26, or a pharmaceutically effective fragment thereof; and a pharmaceutically acceptable carrier.

44. A vaccine comprising (a) the polypeptide according to claim 25, or a pharmaceutically effective fragment thereof, where said polypeptide or fragment thereof is optionally attached to an immunogen which is not PAP or another fragment of PAP, and (b) a pharmaceutically acceptable carrier.

45. A vaccine comprising (a) the polypeptide according to claim 41 or a pharmaceutically effective fragment thereof, where said polypeptide or fragment thereof is optionally attached to an immunogen which is not PAP or another fragment of PAP, and (b) a pharmaceutically acceptable carrier.

46. An immunogenic composition comprising a nucleic acid molecule comprising the nucleic acid sequence according to claim 26 or a pharmaceutically effective fragment thereof, and a pharmaceutically acceptable carrier.

47. A immunogenic composition comprising (a) the polypeptide according to claim 25 or a pharmaceutically effective fragment thereof, where said polypeptide or fragment thereof is optionally attached to an immunogen which is not PAP or another fragment of PAP, and (b) a pharmaceutically acceptable carrier.

48. A immunogenic composition comprising (a) the polypeptide according to claim 41 or a pharmaceutically effective fragment thereof, where said polypeptide or fragment thereof is optionally attached to an immunogen which is not PAP or another fragment of PAP, and (b) a pharmaceutically acceptable carrier.

49. A kit comprising the polypeptide according to claim 25 for use with a method of detecting or monitoring cancer.

50. A kit comprising the nucleic acid molecule according to claim 26 for use with a method of detecting or monitoring cancer.

51. A kit comprising the monoclonal antibody according to claim 29 for use with a method of detecting or monitoring cancer.


SEQUENCE LISTING (13 sequences)

SEQ ID NO: 1 - protein, 9 aa, Homo sapiens
ILLWQPIPV

SEQ ID NO: 2 - protein, 15 aa, Homo sapiens
CPRFQELESETLKSE

SEQ ID NO: 3 - protein, 13 aa, Influenza virus
PKYVKQNTLKLAT

SEQ ID NO: 4 - protein, 13 aa, Hepatitis B virus
TPPAYRPPNAPIL

SEQ ID NO: 5 - protein, 9 aa, Homo sapiens
ALDVYNGLL

SEQ ID NO: 6 - protein, 9 aa, Homo sapiens
VLAKELKFV

SEQ ID NO: 7 - protein, 10 aa, Homo sapiens
IMYSAHDTTV

SEQ ID NO: 8 - protein, 9 aa, Homo sapiens
ILLWQPIPV

SEQ ID NO: 9 - protein, 10 aa, Homo sapiens
ALASCFCFFC

SEQ ID NO: 10 - protein, 15 aa, Homo sapiens
PQGFGQLTQLGMEQH

SEQ ID NO: 11 - protein, 15 aa, Homo sapiens
CPRFQELESETLKSE

SEQ ID NO: 12 - protein, 15 aa, Homo sapiens
SKVYDPLYSESVHNF

SEQ ID NO: 13 - protein, 8 aa, Influenza virus
GILGFVFT